(19) United States
(12) Patent Application Publication
(10) Pub. No.: US 2021/0217419 A1
(43) Pub. Date: Jul. 15, 2021

(54) LIP-LANGUAGE RECOGNITION AAC SYSTEM BASED ON SURFACE ELECTROMYOGRAPHY

(71) Applicant: SHENZHEN INSTITUTES OF ADVANCED TECHNOLOGY CHINESE ACADEMY OF SCIENCES, Shenzhen (CN)

(72) Inventors: Shixiong CHEN, Shenzhen (CN); Mingxing ZHU, Shenzhen (CN); Xiaochen WANG, Shenzhen (CN); Guanglin LI, Shenzhen (CN); Zijian YANG, Shenzhen (CN); Xin WANG, Shenzhen (CN)

(21) Appl. No.: 16/960,496

(22) PCT Filed: Dec. 31, 2019
(86) PCT No.: PCT/CN2019/130814

§ 371 Date: Jul. 7, 2020

(30) Foreign Application Priority Data
Mar. 25, 2019 (CN) 201910228442.4

(51) Int. Cl. G10L 15/25; G10L 15/00; G10L 25/51; G06F 3/01

(52) U.S. Cl. CPC G10L 15/25; G06F 3/015; G10L 25/51; G10L 15/005

(57) ABSTRACT

The present application discloses a lip-language recognition AAC system based on surface electromyography, which includes: a training subsystem configured to collect the facial and neck EMG signals during lip-language movements through the high-density electrode array, improve the signal quality through the signal preprocessing algorithm, classify the lip-language movements through the classification algorithm, select the optimal number of electrodes and optimal positions through the channel selection algorithm, and establish the optimal matching template between the EMG signals and the lip-language information, and upload it to the network terminal for storage; and a detection subsystem configured to collect the EMG signals at the optimal positions during the lip-language movements based on the optimal number and positions of electrodes selected by the training subsystem, call the optimal matching template, classify and decode the EMG signals, recognize the lip-language information, and convert it into corresponding voice and picture information for display in real time.

### TECHNICAL FIELD

The present application pertains to the field of lip-language recognition AAC (Augmentative and Alternative Communication) technologies, in particularly relates to a lip-language recognition AAC system based on surface electromyography (SEMG).

### BACKGROUND

Language is a human-specialized vital ability to express emotions, convey information, and participate in social interactions. Speaking is the foundation of language expression. Moreover, speaking is a very complicated process that the central neural system controls the coordinated movement of muscles, which is a result of the coordination and cooperation of multiple organs and muscle groups. Facial muscles and neck muscles will move accordingly during the speaking process, and the movement patterns of corresponding facial and neck muscles are different regarding different speaking tasks. Therefore, through collection of electrical signals from the surface muscles of the face and neck, and by a way of feature extraction and classification, different speaking tasks can be matched with different electrophysiological changes of the muscle groups, such that the speaking information can be recognized, thereby assisting patients to communicate with others.

According to the results of the second sample survey of Chinese disabled persons in 2006, there were 82.96 million disabled people in China, including 1.27 million speaking-disabled people who accounted for 1.53% of the total population. Speaking disorder severely reduces their life quality, affects their daily life communication, and causes inconvenience in communication, which carries a heavy burden for their families and society. The diagnosis and treatment of the speaking disorder are still not mature enough clinically, therefore the speaking disabled persons urgently need an AAC product to assist them express and communicate.

The surface myoelectric signal is a one-dimensional voltage-time sequence signal, which is acquired after a bioelectrical change generated during a voluntary activity or an involuntary activity of the muscular system is guided by a surface electrode, amplified, displayed and recorded. It reflects a sum in time and space of potentials of a lot of peripheral motor units generated by bioelectrical activities of motor neurons, has a relatively high correlation with muscle activities, and can reflect activation level of related muscles to a certain extent. Therefore, the movement condition of related muscles can be observed by analyzing the SEMG. The SEMG, as an objective and quantizable means, has the advantages of non-invasiveness, simple operation, low cost, and providing quantitative and deterministic analysis, so it is widely used in fields such as medical research, human-computer interaction, etc.

In recent years, there are already some studies using the SEMG for speech recognition. However, in the prior art, the SEMG signals were acquired by using only a few electrodes placed on a few known speaking-related muscles, the number and positions of the electrodes were individually subjective selected, and the number of selected electrodes and the number of channels might not be the optimal solution. Therefore, there is a specific limitation leading to relatively low accuracy of lip-language recognition.

### Technical Problems

Given this, embodiments of the present application provide a lip-language recognition AAC system based on SEMG for patients who have difficulty in speaking but can express through shapes of their mouth or lip-language, so as to solve the problems in the prior art that it was difficult to obtain the optimal solution by individually and subjectively selecting the number and positions of the electrodes and the accuracy of speech signal recognition was relatively low.

### SUMMARY

The embodiments of the present application provide a lip-language recognition AAC system based on SEMG, which includes:

a training subsystem, configured to collect EMG signals in face and neck during a lip-language movement through a high-density electrode array, improve signal quality through a signal preprocessing algorithm, classify the type of the lip-language movement through a classification algorithm, select an optimal number and positions of the electrodes through a channel selection algorithm, establish an optimal matching template between the EMG signals and lip-language information, and upload the optimal matching template to a network terminal for storage;

a detection subsystem, configured to collect the EMG signals at the optimal positions during the lip-language based on the optimal number and positions of the electrodes selected by the training subsystem, call the optimal matching template, classify and decode the EMG signals, recognize the lip-language information, and transform the lip-language information into corresponding voice and picture information displayed in real-time, thereby achieving the lip-language recognition.

Further, the training subsystem includes a slave computer of the training subsystem and a principal computer of the training subsystem, and the slave computer of the training subsystem includes:

a high-density electrode array, configured to obtain the high-density EMG signals of a speaking-relevant muscle group during the user perform lip-language tasks by being pasted on the speaking-relevant muscle group of the face and neck;

an EMG collection module, configured to perform amplification, filtering, and analog-to-digital conversion on the signals collected by the high-density electrode array, and transmit the processed signals to the principal computer of the training subsystem.

Further, the principal computer of the training subsystem includes a user interaction module, and a training module for signal classification, correction and matching feedback, wherein the user interaction module includes:

an EMG signal display sub-module, configured to display the collected EMG signals in real time;

a lip-language training scene display sub-module, configured to provide lip-language scene pictures and texts;

a channel selection and positioning chart display sub-module, configured to provide distribution of the electrodes positioned on the face and neck.

Further, the training module for signal classification, correction, and matching feedback includes:

a signal processing sub-module, configured to filter out power-line interference and baseline shift by using a filter, and filter out interference noise from the EMG signals by using a wavelet transform and a template matching algorithm;

a classification sub-module, configured to extract the EMG signals related to pronunciation of a specified short sentence, extract a feature value, establish a corresponding relationship between the EMG signals and the specified short sentence, and classify the collected lip-language contents based on EMG information;

a channel selection sub-module, configured to select the optimal matching template, create a personal training set, and transfer the optimal matching template and the personal training set to the network terminal.

Further, the detection subsystem includes a slave computer of the detection subsystem and a principal computer of the detection subsystem, and the slave computer of the detection subsystem includes:

an SMD (Surface Mounted Device) flexible electrode, configured to collect the EMG signals at the optimal positions during the lip-language movement;

a wireless EMG collection module, configured to wirelessly transmit EMG information collected by the SMD flexible electrode to the principal computer of the detection subsystem.

Further, the principal computer of the detection subsystem includes:

a personal training set download module, configured to call a personal training set from a network shared port of the training subsystem by connecting to the network, and store the personal training set in an APP client terminal;

a lip-language information recognition and decoding module, configured to denoise and filter the signals, perform feature matching for the EMG signals and the personal training set, decode the lip-language information and recognize lip-language contents by using a classification algorithm, convert the lip-language contents corresponding to a classification result into text information and into voice and pictures for transmission and display in real time;

an APP display and interaction module, configured to display channel selection and an optimal data set, show the positions of the electrodes in real time, display the EMG signals in real time, display the classification result in real time, and/or display the voice, the pictures and translation.

Further, the lip-language information recognition and decoding module is further configured to transmit the recognition result to an emergency contact set by the system.

Further, the high-density electrode array comprises 130 electrodes, and the electrodes are arranged in a high-density form with a center-to-center spacing of 1 cm.

Further, the slave computer of the training subsystem further includes an orifice plate for arranging the electrodes.

Further, the EMG collection module includes an MCU, an analog-to-digital converter, an independent synchronous clock, a signal filtering preamplifier, and a low-noise power supply.

### Beneficial Effects

Compared with the prior art, the beneficial effects of the embodiments of the present application lie in that: the embodiments of the present application use the training subsystem to collect the facial and neck EMG signals during lip-language movements through the high-density electrode array, improve the signal quality through the signal preprocessing algorithm, classify the lip-language movements through the classification algorithm, select the optimal number of electrodes and optimal positions through the channel selection algorithm, and establish the optimal matching template between the EMG signals and the lip-language information, and upload it to the network terminal for storage. On this basis, the detection subsystem is used to collect the EMG signals at the optimal positions during the lip-language movements based on the optimal number and positions of electrodes selected by the training subsystem, call the optimal matching template, classify and decode the EMG signals, recognize the lip-language information, and convert it into corresponding voice and picture information for display in real-time, thereby realizing the lip-language recognition. Through this strategy of being first comprehensive and then partial, the EMG signals during the pronunciation process are acquired by using the high-density electrode array in real-time and completely, and the electrodes that contribute the most to the lip-language movements during the muscle activity are selected after processing and analyzing, and the optimal number of electrodes and electrode positions are determined to realize objective determination of selection for the lip-language recognition electrodes, thus significantly improving the accuracy of the lip-language recognition.

### DESCRIPTION OF THE DRAWING

In order to more clearly illustrate the technical solutions in the embodiments of the present application, the drawing required to be used for describing the embodiments or the prior art is briefly introduced below. Obviously, the drawing in the following description only shows some embodiments of the present application, and other drawings may be obtained without creative work based on the drawing for those of ordinary skill in the art.

FIG. 1 is a structural block diagram of the lip-language recognition AAC system based on surface electromyography provided by an embodiment of the present application.

### EMBODIMENTS OF THE APPLICATION

In order to make the purposes, characteristics, and advantages of the present application more obvious and understandable, the technical solutions in the embodiments of the present application will be described clearly and completely in conjunction with the drawing in the embodiments of the present application. Obviously, the described embodiments are only a part of the embodiments but not all the embodiments of the present application. Based on the embodiments of the present application, all other embodiments obtained without creative work by those of ordinary skill in the art fall within the protection scope of the present application.

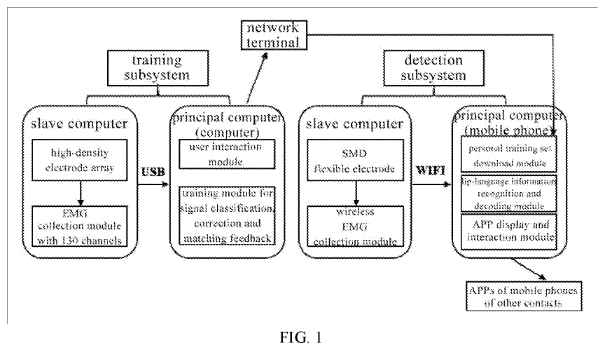

FIG. 1 shows a structural block diagram of the lip-language recognition AAC system based on surface electromyography provided by an embodiment of the present application. For ease of description, only parts related to this embodiment are shown.

Referring to FIG. 1, the lip-language recognition AAC system based on surface electromyography provided by this embodiment of the present application may include a training subsystem and a detection subsystem.

The training subsystem is configured to collect facial and neck EMG signals during a lip-language through a high-density electrode array, improve signal quality through a signal preprocessing algorithm, classify the type of the lip-language movement through a classification algorithm, select the optimal number and the optimal positions of the electrodes through a channel selection algorithm, establish an optimal matching template between the EMG signals and lip-language information, and upload the optimal matching template to a network terminal for storage.

The detection subsystem is configured to collect the EMG signals at the optimal positions during the lip-language movement based on the optimal number and positions of the electrodes selected by the training subsystem, call the optimal matching template, classify and decode the EMG signals, recognize the lip-language information, and transform the lip-language information into corresponding voice and picture information displayed in real time, thereby achieving the lip-language recognition.

The training subsystem may include a slave computer and a principal computer, that is, the slave computer of the training subsystem and the principal computer of the training subsystem.

The slave computer of the training subsystem may include a high-density electrode array and an EMG collection module.

The high-density electrode array is configured to obtain high-density EMG signals of a speaking-related muscle group during the user perform lip-language speaking by being pasted on the speaking-related muscle group of the face and neck. The reasons why the high-density EMG signals are required to be obtained first by the high-density electrode array lie in that, everyone's habits and pronunciation manners are not entirely the same, everyone's parts applying force to pronounce are not entirely the same, there is a certain difference in muscle activities for everyone during the pronunciation, and everyone's characteristics and positions of the muscle activities are also different, so it is very unreasonable to place the electrodes at positions of the same several muscles for different people. Therefore, in this embodiment, the high-density electrode array is first used to collect comprehensive EMG signals.

The high-density electrode array may be composed of a large number of electrodes, and the specific number of the electrodes and the spacing between the electrodes may be customized according to the size of the user's face and neck, so as to ensure that the comprehensive EMG signals from the speaking-related muscle group are collected. Preferably, the high-density electrode array may include 130 electrodes, and the electrodes are arranged in a high-density form with a center-to-center spacing of 1 cm.

The EMG collection module may be an EMG collection module provided with 130 channels, and may include an MCU (Micro Controller Unit), an analog-to-digital converter, an independent synchronous clock, a signal filtering preamplifier, and a low-noise power supply. The EMG collection module is configured to perform amplification, filtering, and the analog-to-digital conversion on the signals collected by the high-density electrode array, and transmit it to the principal computer of the training subsystem through a USB or other transmission paths.

Preferably, the slave computer of the training subsystem may further include an orifice plate for arranging the electrodes, and each orifice plate is provided with corresponding hole sites for the electrodes, and the hole spacing is about 1 cm to ensure that the distance among the electrodes is small enough. The orifice plate is divided into 4 specifications: 20 holes, 25 holes, 40 holes, and 48 holes, which are capable of respectively arranging 20 electrodes, 25 electrodes, 40 electrodes, and 48 electrodes at the same time, thereby reducing workload and making operations more convenient.

The principal computer of the training subsystem may be a device such as a desktop computer, a notebook computer, and a tablet computer etc, which includes a user interaction module, and a training module for signal classification, correction and matching feedback.

The user interaction module may include an EMG signal display sub-module, a lip-language training scene display sub-module, and a channel selection and positioning chart display sub-module.

The EMG signal display sub-module is configured to display the collected EMG signals in real-time and provide a selection function for a single-channel signal, such that the signal quality in all channels can be observed in real-time, and the reliability of the signals is ensured.

The lip-language training scene display sub-module is configured to provide lip-language scene pictures and texts required in daily life and providing a personalized training set for the user, collect the EMG signals by training at a fixed scene mode and store the EMG signals as a lip-language analysis EMG database. In addition, this sub-module further provides task prompts such as “read again”, “next scene”, etc., to provide friendly interaction for repeated training and a next operation.

The channel selection and positioning chart display sub-module is configured to provide distribution of the electrodes positioned on the face and neck, and display the number and specific positions of the selected effective channels in real time through training classification.

The training module for signal classification, correction and matching feedback may include a signal processing sub-module, a classification sub-module and a channel selection sub-module.

The signal processing sub-module is configured to preliminarily filter out power-line interference and baseline shift by using an IIR band-pass filter and a filter based on an optimization algorithm, and then further filter out algorithm interference noise such as an artifact and electrocardio etc. from the EMG signals by using an algorithm such as wavelet transform and a template matching algorithm, and preprocess the signals to improve signal quality and reliability.

The classification sub-module is configured to perform algorithm processing such as normalization and blind source separation on the signals to extract the EMG signals related to the pronunciation of a specified short sentence, extract a feature value, establish a corresponding relationship between the EMG signals and the specified short sentence by using a linear classifier, a neural network and an SVM (Support Vector Machine) technology, and classify the collected lip-language contents based on the EMG information.

The channel selection sub-module is configured to select an EMG template with the minimum number of channels and the optimal classification accuracy after multiple corrections and matches, store and save the optimal matching template of the EMG signals and the lip-language information, create a personal training set, and transfer the optimal template data set to the network terminal.

Since everyone's habits and pronunciation manners are not entirely the same, everyone's parts applying force to pronounce are not entirely the same, there is a certain difference in muscle activities for everyone during the pronunciation, and everyone's characteristics and positions of the muscle activities are also different. Therefore, in order to accurately recognize the lip-language information, it is necessary to do multiple pronunciation training for the user, create a personal training set, store the corresponding relationship between the EMG signals and the specified short sentence, and determine the personalized optimal solution of the electrodes.

The detection subsystem may include two parts including a slave computer and a principal computer, namely, a slave computer of the detection subsystem and a principal computer of the detection subsystem.

The slave computer of the detection subsystem includes an SMD flexible electrode and a wireless EMG collection module.

The SMD flexible electrode is configured to collect the EMG signals at the optimal position in a lip-language movement. The existing EMG electrodes are electrodes made of a hard board, and have a limited degree of fitness with the skin, thus the pulling and deformation of the skin is likely to cause relatively large noise interference to the EMG data. The SMD flexible electrode includes an electrode made of an FPC soft board containing several flexible materials and forms a customized flexible electrode slice that is bendable and tightly fitted with the skin, and the specific number of electrodes may be set according to the actual situation. Preferably, the specific number of electrodes may be set as 8. The user selects the number of flexible electrodes required to be used and the placement positions of the electrodes on the face and neck according to a calculation result of the training subsystem. The SMD flexible electrode has a high degree of personalization, is close to the skin, and slightly deforms with the skin. Therefore the acquired EMG information is more stable and reliable.

The wireless EMG collection module integrates functions of 8-channel EMG signal collection and wireless transmission, in which a microcontroller integrating a WIFI function, a preamplifier circuit, an analog-to-digital conversion circuit, etc. are used to wirelessly transmit the EMG information collected by the SMD flexible electrodes to the principal computer of the detection subsystem through WIFI and the like. The wireless transmission is more convenient than traditional wired electrodes, since it is simple to wear, and reduces the influence of winding between the wired electrode wires. The WIFI transmission does not lose data, ensuring data integrity. The multi-channel EMG information is transmitted wirelessly at the same time, which makes up for the defect of insufficient information in the traditional method due to fewer electrode channels.

The principal computer of the detection subsystem may be a device such as a mobile phone, a tablet computer etc., which includes a personal training set download module, a lip-language information recognition and decoding module and an APP display and interaction module.

The personal training set download module is configured to call a personal training set from a network shared port of the training subsystem by connecting to the network and store it in an APP client terminal.

The lip-reading information recognition and decoding module includes functional modules such as a data preprocessing module, an online myoelectricity classification module, a classification result voice conversion module etc., and is configured to denoise and filter the signals by using the IIR filter, the wavelet transform, etc., and perform feature matching for the EMG signals and the personal training set, decode the lip-language information and recognize the lip-language contents by using the classification algorithm, convert the lip-language contents corresponding to the classification result into text information and into voice and pictures for transmission and display in real-time through processing the called voice and picture template. The lip-language information recognition and decoding module is further configured to transmit the recognition result to an emergency contact set by the system through the APP.

Most of the current AAC systems require the communicator and the patient to be face-to-face or stay close. Nevertheless, in daily life, the patient also needs to communicate with others on many occasions when he is alone, such as asking for help at home alone. This embodiment uses the wireless transmission technology to recognize the patient's lip-language information, on the one hand, the lip-language recognition result is converted into the voice and pictures through the APP for broadcast and display; on the other hand, the lip-language recognition result is automatically sent to the APP of the mobile phone of the set emergency contact through a user link, such that others can obtain the patient's lip-language information instantly and remotely.

The APP display and interaction module is configured to display channel selection and an optimal data set, display positions of the electrodes in real time, display the myoelectric signals in real time, display the classification result in real time, and/or display the voice, the picture and the translation.

The above contents describe the collection and analysis for the EMG information of the speaking-related muscle groups of the face and neck. Besides, the muscles of other parts related to the pronunciation function, such as the abdomen, also contain certain pronunciation movement information, which may also be a source of the EMG information in this embodiment to recognize the pronunciation information.

The core contents of this embodiment lie in lip-language recognition based on the high-density EMG. The lip-language recognition can not only be used for people with the speaking disorder but also be promoted to other occasions where pronunciation is inconvenient or noise is relatively loud, such as underwater operations, noisy factories, etc. Therefore, lip-language recognition is provided with colossal development.

In summary, the embodiments of the present application use the training subsystem to collect the facial and neck EMG signals during lip-language movements through the high-density electrode array, improve the signal quality through the signal preprocessing algorithm, classify the lip-language movements through the classification algorithm, select the optimal number of electrodes and optimal positions through the channel selection algorithm, and establish the optimal matching template between the EMG signals and the lip-language information, and upload it to the network terminal for storage. On this basis, the detection subsystem is used to collect the EMG signals at the optimal positions during the lip-language movements based on the optimal number and positions of electrodes selected by the training subsystem, call the optimal matching template, classify and decode the EMG signals, recognize the lip-language information, and convert it into corresponding voice and picture information for display in real time, thereby realizing the lip-language recognition. Through this strategy of being first comprehensive and then partial, the EMG signals during the pronunciation process are acquired by using the high-density electrode array in real-time and thoroughly, and the electrodes that contribute the most to the lip-language movements during the muscle activity are selected after processing and analyzing, and the optimal number of electrodes and electrode positions are determined to realize objective determination of selection for the lip-language recognition electrodes, thus significantly improving the accuracy of the lip-language recognition.

Those skilled in the art can clearly understand that, for the convenience and conciseness of description, only divisions of the above-mentioned functional systems or modules are used as examples for illustration. In practical applications, the functions mentioned above can be allocated to different functional systems or modules to execute according to needs, so as to complete all or part of the functions described above. The functional systems or modules in the embodiments may be integrated into one processing unit; alternatively, each unit may exist alone physically; alternatively, two or more units may be integrated into one unit. The above-integrated unit may be implemented in a form of hardware, or may be implemented in a form of a software functional unit. In addition, the specific names of each functional system and module are only used for distinguishing each other, and are not used to limit the protection scope of the present application.

The embodiments mentioned above are only used to illustrate the technical solutions of the present application, but not to limit them. Although the present application has been described in detail with reference to the foregoing embodiments, those of ordinary skill in the art should understand that they can still modify the technical solutions described in the foregoing embodiments, or equivalently replace some of the technical features thereof. These modifications or replacements do not deviate the nature of corresponding technical solutions from the spirit and scope of the technical solutions of the embodiments of the present application, and should be included within the protection scope of the present application.

What is claimed is:

1. A lip-language recognition AAC system based on surface electromyography, comprising:
a training subsystem, configured to collect facial and neck EMG signals in a lip-language movement through a high-density electrode array, improve signal quality through a signal preprocessing algorithm, classify the type of the lip-language movement through a classification algorithm, select an optimal number and optimal positions of the electrodes through a channel selection algorithm, establish an optimal matching template between the EMG signals and lip-language information, and upload the optimal matching template to a network terminal for storage; and
a detection subsystem, configured to collect the EMG signals at the optimal positions during the lip-language movement based on the optimal number and the optimal positions of the electrodes selected by the training subsystem, call the optimal matching template, classify and decode the EMG signals, recognize the lip-language information, and transform the lip-language information into corresponding voice and picture information for real-time display, thereby achieving lip-language recognition.

2. The system according to claim 1, wherein the training subsystem comprises a slave computer of the training subsystem and a principal computer of the training subsystem, and the slave computer of the training subsystem comprises:
a high-density electrode array, configured to obtain the high-density EMG signals of a speaking-related muscle group during user lip-language by being pasted on the speaking-related muscle group of the face and neck; and
an EMG collection module, configured to perform amplification, filtering, and analog-to-digital conversion on the signals collected by the high-density electrode array, and transmit the processed signals to the principal computer of the training subsystem.

3. The system according to claim 2, wherein the principal computer of the training subsystem comprises a user interaction module, and a training module for signal classification, correction and matching feedback, wherein the user interaction module comprises:
an EMG signal display sub-module, configured to display the collected sEMG signals in real-time;
a lip-language training scene display sub-module, configured to provide lip-language scene pictures and texts; and
a channel selection and positioning chart display sub-module, configured to provide distribution of the electrodes positioned on the face and neck.

4. The system according to claim 3, wherein the training module for signal classification, correction and matching feedback comprises:
a signal processing sub-module, configured to filter out power-line interference and baseline shift by using a filter, and filter out interference noise from the EMG signals by using a wavelet transform and a template matching algorithm;
a classification sub-module, configured to extract the EMG signals related to speaking of a specified short sentence, extract a feature value, establish a corresponding relationship between the EMG signals and the specified short sentence, and classify collected lip-language contents based on EMG information;
a channel selection sub-module, configured to select the optimal matching template, create a personal training set, and transfer the optimal matching template and the personal training set to the network terminal.

5. The system according to claim 1, wherein the detection subsystem comprises a slave computer of the detection subsystem and a principal computer of the detection subsystem, and the slave computer of the detection subsystem comprises:
an SMD flexible electrode, configured to collect the EMG signals at the optimal positions during the lip-language movement; and
a wireless EMG collection module, configured to wirelessly transmit EMG information collected by the SMD flexible electrode to the principal computer of the detection subsystem.

6. The system according to claim 5, wherein the principal computer of the detection subsystem comprises:
a personal training set download module, configured to call a personal training set from a network shared port of the training subsystem by connecting to the network, and store the personal training set in an APP client terminal;
a lip-language information recognition and decoding module, configured to denoise and filter the signals, perform feature matching for the EMG signals and the personal training set, decode the lip-language information and recognize lip-language contents by using a classification algorithm, convert the lip-language contents corresponding to a classification result into text information and into voice and pictures for transmission and display in real time; and
an APP display and interaction module, configured to display channel selection and an optimal data set, display the positions of the electrodes in real time, display the EMG signals in real time, display the classification result in real time, and/or display the voice, the pictures and translation.

7. The system according to claim 6, wherein the lip-language information recognition and decoding module is further configured to transmit the recognition result to an emergency contact set by the system.

8. The system according to claim 1, wherein the high-density electrode array comprises 130 electrodes, and the electrodes are arranged in a high-density form with a center-to-center spacing of 1 cm.

9. The system according to claim 2, wherein the slave computer of the training subsystem further comprises an orifice plate for arranging the electrodes.

10. The system according to claim 2, wherein the EMG collection module comprises an MCU, an analog-to-digital converter, an independent synchronous clock, a signal filtering preamplifier, and a low-noise power supply.
